(19) United States
(12) Patent Application Publication
(10) Pub. No.: US 2004/0049132 A1
(43) Pub. Date: Mar. 11, 2004

(54) Device for body activity detection and processing

(75) Inventors: Bradford Scott Barron, Cincinnati, OH (US); Emily Charlotte Boswell, Isleworth (GB); Corinne Dauger-Strauss, Brussels (BE); Joseph Fernand Deflander, Wespelaar (BE); Neil Archibald MacGilp, Bramley (GB); Chris Van den Wouwer, Deurne (BE); David Keith Ewart, Uxbridge (GB); Derk-Jan Dijk, Guildford (GB)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH

(21) Appl. No.: 10/314,842

(22) Filed: Dec. 9, 2002

Related U.S. Application Data
(63) Continuation of application No. PCT/US01/19054, filed on Jun. 14, 2001.
(63) Continuation of application No. PCT/US01/19056, filed on Jun. 14, 2001.
(63) Continuation of application No. PCT/US01/19055, filed on Jun. 14, 2001.

(30) Foreign Application Priority Data
Jun. 15, 2000 (EP) 00112681.2
Jun. 15, 2000 (EP) 00112680.4

(51) Int. Cl. A61B 5/103

(52) U.S. Cl. 600/595

(57) ABSTRACT

A method and device for monitoring a body activity. The device has an actimetry sensor for measuring the activity and storage means for receiving data from the actimetry sensor. The data are analysed according to a method using summation algorithm, where a plurality of parameters relating to the activity are summed to provide advisory information relating to that activity. The analysis may include pre-programmed biasing constants or user supplied biasing constants.

### CROSS REFERENCE TO PRIOR APPLICATIONS

This is a continuation of International Application PCT/US01/1 9054, with an international filing date of Jun. 14, 2001, published in English; International Application PCT/US01/1 9056, with an international filing date of Jun. 14, 2001, published in English; and International Application PCT/US01/19055, with an international filing date of Jun. 14, 2001, published in English.

### FIELD OF INVENTION

This invention relates to the detection of body activity, such as sleep patterns, and the analysis of data related to such functions for a user.

Background of the Invention

In recent years there has been much study of body functions, such as sleep activity, and associated analysis of the relevance of such functions to the general health of the body and the body's need for appropriate body functions (such as sleep patterns) to occur on a regular basis for adequate periods of time. As part of this research numerous devices have been proposed to assist in such measurement and analysis.

For example, WO-A-9714354 discloses a device and corresponding method which collects data for analysing sleep disturbances so that such data can be interpreted by a specialist at a future date.

However, this type of device requires operation by a highly skilled user and provides analysis which is difficult to interpret by anybody other than a specialist, as well as being expensive and sometimes unreliable. Furthermore, this device is unable to provide a detailed history over an extended time period for an individual.

Other systems are uncomfortable, cannot be worn for extended periods and/or cannot be worn without restricting body movement.

### SUMMARY OF THE INVENTION

The present invention is a device for monitoring body activity, the device comprising an actimetry sensor for measuring body activity, storage means for receiving data from the actimetry sensor, means for analysing the data to provide advisory information, and means for displaying the advisory information to a user.

The actimetry sensor may be an accelerometer or may be a simple motion sensor or tilt switch. The body activity being monitored may be sleep, and/or waking activity.

The storage means may store data from the actimetry sensor together with temporal information. In such a case, the means for analysing the stored data may provide processing based upon both body activity information and temporal information to provide advisory information to the user.

The advisory information provided to the user may include an indication of the quality of the activity, such as the quality of the sleep, whether or not the duration of the activity is sufficient, an indication as to whether the total amount of the activity over an extended period is acceptable, as well as other data related to other long term body activity, for example. The device can be configured to detect activity during the day. The body activity that is measured can, as well as being actual time slept, be the number of awakenings, an indication as to how intermittent the sleep was, time taken before sleep, the number of and length of sleep interruptions, sleep proficiency, the number minutes immobile/moving, etc. Part or all of this information can be provided to identify the least and most active times during the day.

The device may include an input (such as buttons) for receiving input data from a user, such as desired time to go to sleep, the need to awake early for a particular event, as well as possible information relating to the age of the user, their sex, as well as, optionally, additional information such as what they perceive their energy level to be.

The device may have one or more additional sensors to also measure body pulse rate variability, blood pressure variability or other body activities such as eyelid movement or respiration. In this case, sleep phases such as REM, slow light sleep, slow deep sleep, or paradoxical sleep may be monitored.

The device may be configured in the style of a wrist watch, and may be arranged to provide additional information to a user, such as time and date information. The device may have an alarm. An additional sensor may be included for detecting data relating to the environment in which the body is placed.

### BRIEF DESCRIPTION OF THE DRAWINGS

One example of the present invention will now be described with reference to the accompanying drawings, in which:

FIG. 1 is a schematic perspective view of a device according to the present invention;

FIG. 2 is a schematic diagram showing some of the functionality of the device of FIG. 1;

FIGS. 3 to 6 are diagrams showing two possible displays from the device of FIG. 1; and

FIG. 7 is a schematic diagram of the internal components of the device of FIG. 1.

### DETAILED DESCRIPTION OF THE DRAWINGS

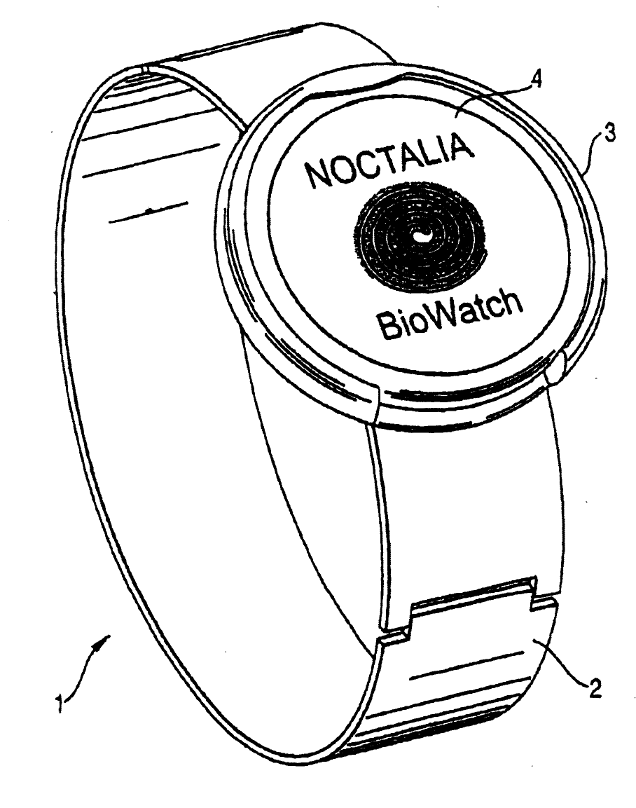

Referring to FIG. 1, a device 1 according to the present invention is, in this example, configured as a wrist watch-style device (although could have a different configuration), with a strap 2 and a component-containing housing 3. On the outer surface of the housing 3 is a display 4, which, in this case, is a liquid crystal display, although a plasma display, etc. could be used.

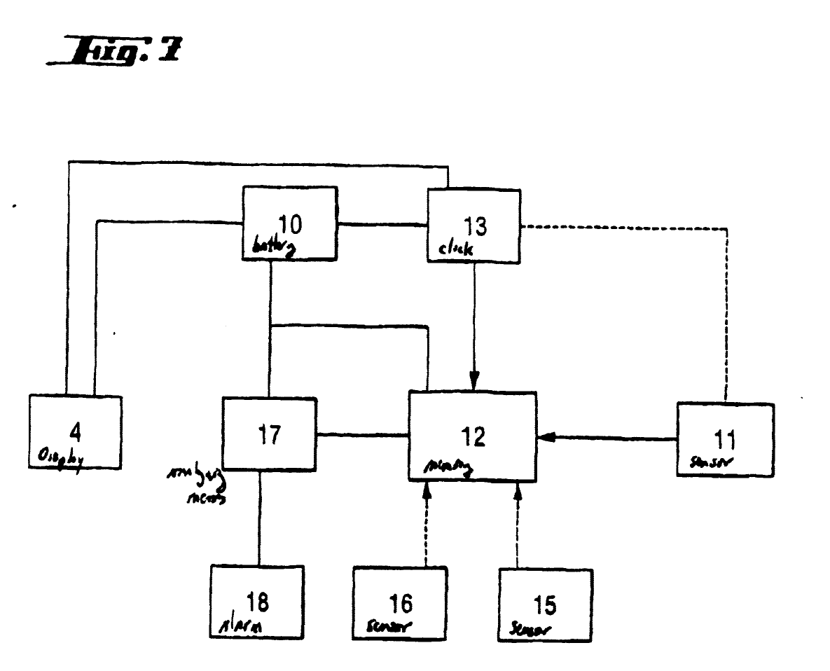

Referring to FIG. 7, a device 1 of FIG. 1 has a number of internal components. The device 1 is powered by a battery 10 (or another form of power supply) which supplies power to the other components of the device 1. An actimetry sensor 11 detects motion in the device 1 and hence motion in the body to which the device 1 is attached in use. The data from the actimetry sensor 1 1 is passed to a memory 12. A clock 13 also provides temporal data to the memory 12 and to the actimetry sensor 11 if necessary, as well as optionally to a display 4. In addition to the actimetry sensor 11 the device 1 may further comprise additional sensors 15, 16, which may detect blood pressure, pulse rate variability etc. Data from these additional optional sensors 15, 16 may also be forwarded to the memory 12. Data from the memory 12 can be requested from analysing means 17, either on a regulated intermittent, continuous, or on a user-requested basis.

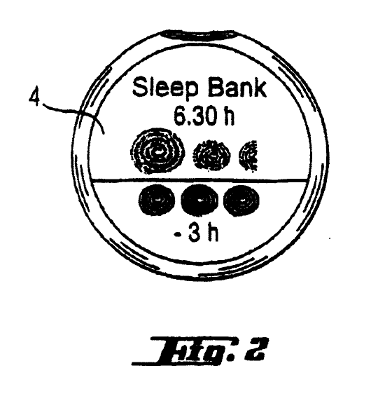

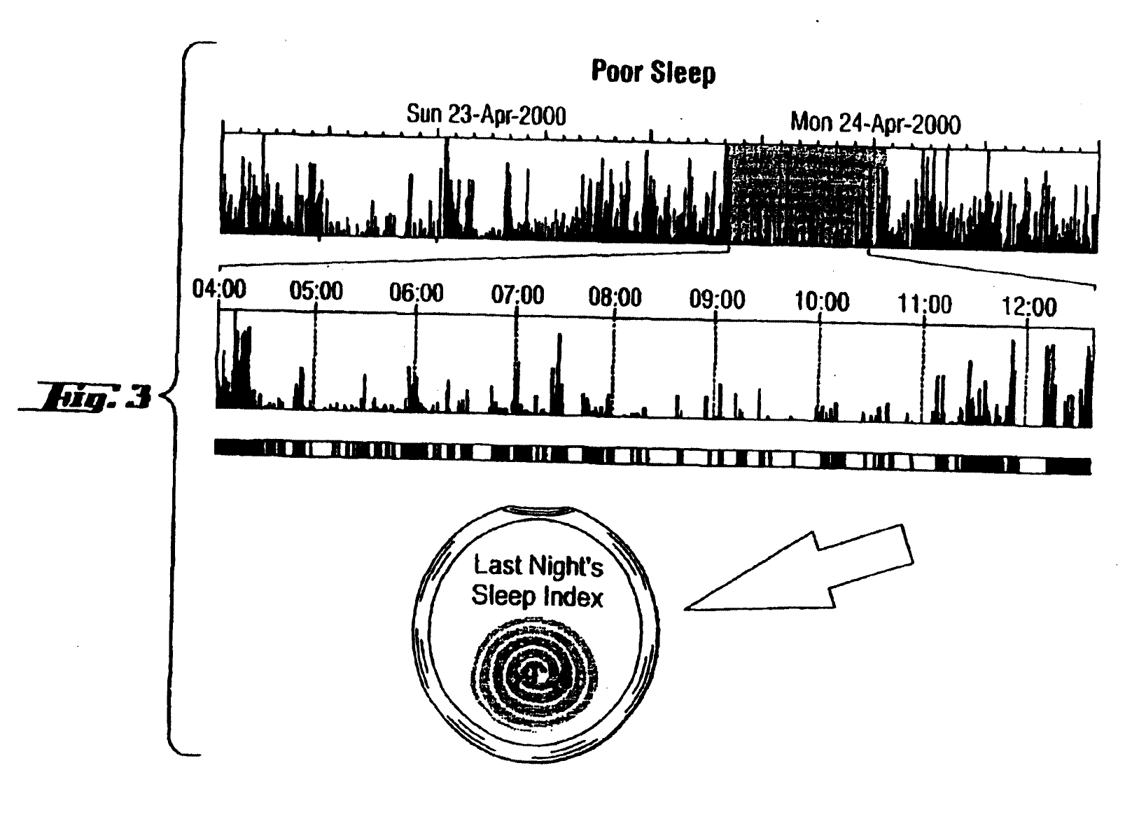

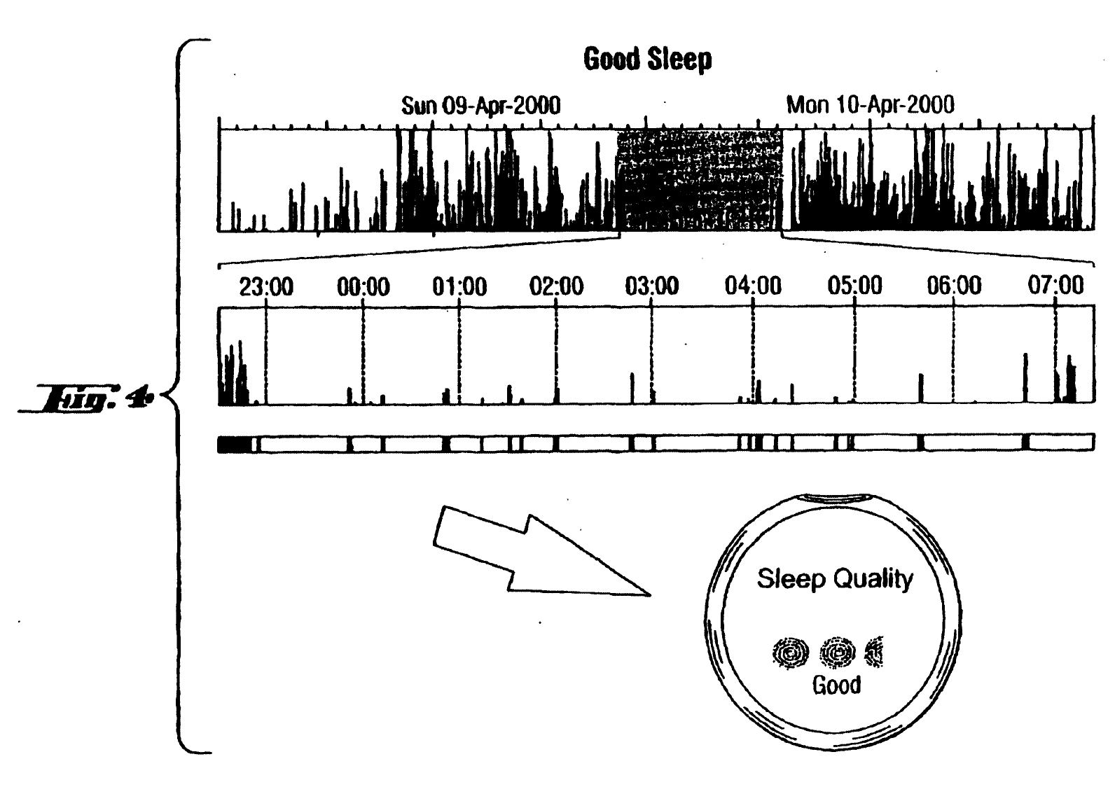

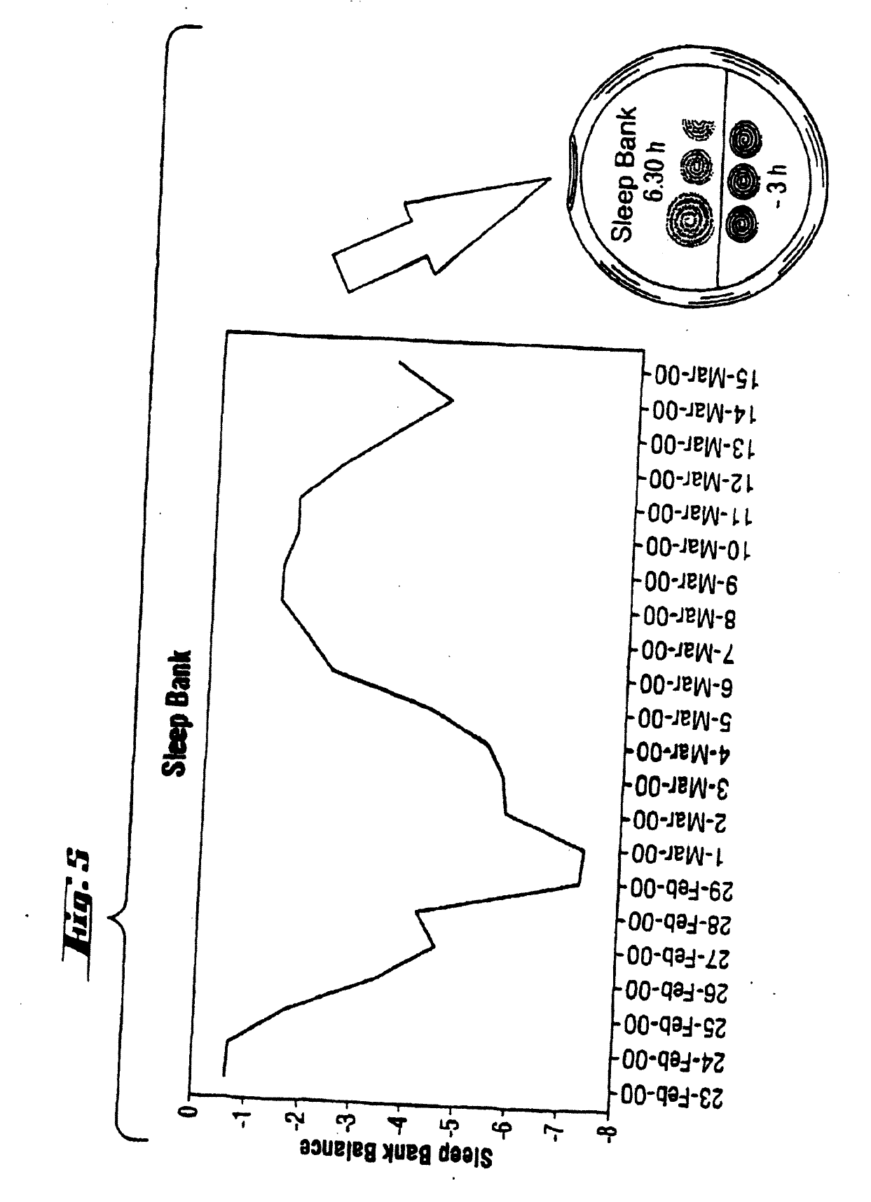

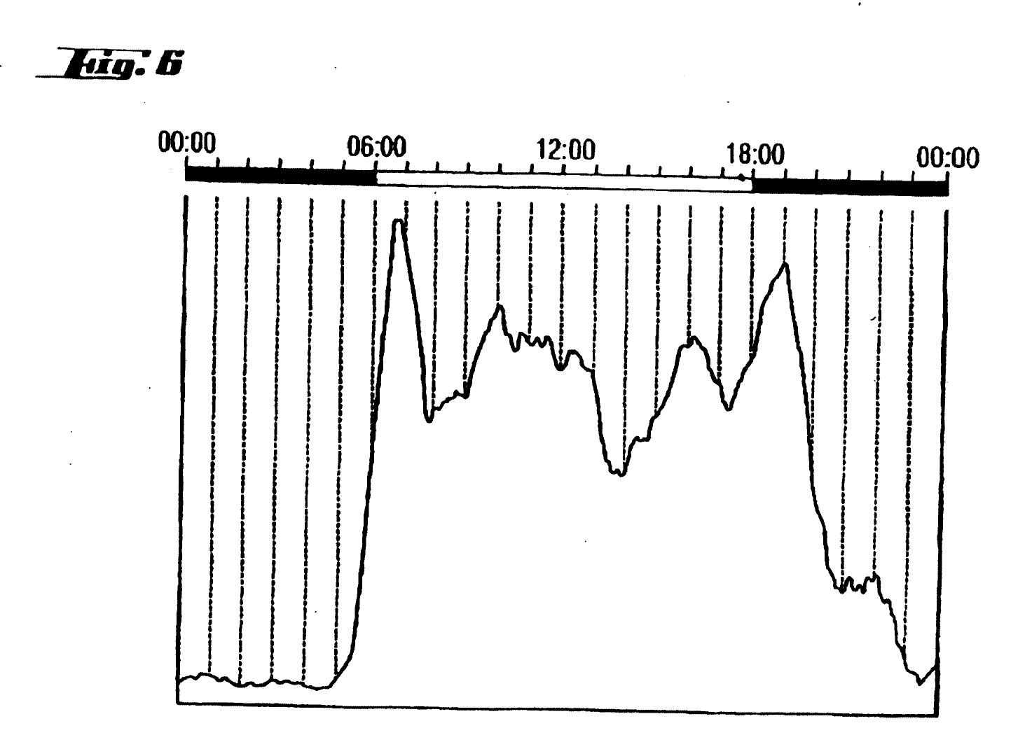

A wide variety of different forms of analysis may be performed by the analysing means 17. Examples of the types of analysis that may be performed will now be described with reference to FIGS. 2 to 6.

The actimetry sensor 11 may provide information in relation to sleep duration and the type of sleep to the analysing means 17. By type of sleep this may include the duration of sleep, number of interruptions, motion during sleep, for example. This information can be analysed by the analysing means 17 to provide information to the display form in terms simply of the total number of hours of effective sleep obtained, although it may provide additional information in relation to the quality of the sleep and the expected value of that sleep in terms of an “energy bank”. By using data stored in the memory 12 over a number of days, weeks or months, the analysing means 17 may also provide information indicative of accumulated sleep deficit or sleep excess. As mentioned above, the data can be provided to a user as and when requested, and is arranged to be provided in a very simple format so that it does not need complex interpretation.

The analysing means 17 may employ a sleep scale such as the Stanford sleep scale in order to score the monitored sleep and receive relevant information from the user. The scale defines different levels of sleepiness as follows:

1—feeling active, vital, alert, wide awake.

2—functioning at a high level, not at peak.

3—relaxed, not full alertness, responsive.

4—a little soggy, not at peak, let down.

5—tired, losing interest, slowed down.

6—drowsy, prefer to be lying down.

7—almost in a reverie, hard to stay awake.

This scale can be shown to a user so that the user can input an indication of how tired they consider themselves to be. For example, the user could be prompted to input an indication as to how they feel when they wake up, with an indication as to the reasons for their feelings being provided by the analysing means 17 from the data collected.

In another example, such an input could be employed during the initial weeks of employing the device to help the device determine whether or not the user is sleeping for the right amount of time to them. For example, on the first day of wearing the device, the device may prompt the user to indicate how much sleep they consider they need. It could then provide information as to the average sleep requirement for someone of their age and sex. However, as the requirements vary from user to user, the device can then monitor sleep over a given period and prompt the user for feedback, not only at that time but also during the day in order to form a sleep diary in the memory of the device. The device may then be configured to adapt the indications that it gives the user based upon the feedback and wake the user at the appropriate time, and then employ a sleep bank once the user's particular requirements have been determined.

The device 1 may have an alarm 18, which can be used simply to wake the user, in the manner of a normal wrist watch alarm, although it may be activated by the analysing means 17 (in conjunction with a heart rate monitor), when it is detected that an appropriate type of sleep is occurring to ensure gentle waking of the user or waking at a time such that they have less sleep inertia.

If additional sensors 15, 16 are provided then additional analysis can be performed dependent upon the type of sensor. If the sensors detect parameters external to the body, such as light, location, sound, air temperature, humidity, barometric pressure, then this information may be compared with information relating to body activity in order to adjust their information. If the sensors determine additional body activity, and detect one or more of muscle tonus, skin temperature, galvanic skin response, etc., then additional analysis of the quality of the sleep may be provided. As a further example, if a blood pressure sensor is employed then additional indications related to general levels of health and activity not specifically related to sleep alone can be provided by the analysing means. If a pulse rate variability detector is employed then this can assist in determining the type of sleep detected and analyzing the quality of that sleep, and can provide further information in relation to whether an acceptable level of aerobic exercise has been performed within the allotted time period, whether it be a day, a week or a month, for example.

If the device 1 provides some form of “sleep bank” indication over a period of time, then the sleep bank may calculate the information to be provided to the user by including a formula such as:

sleep bank (i)=sleep bank (i−1)+(sleep (i)−need) where sleep bank is accumulative of sleep balance on day i, sleep is sleep achieved on the night before day i and need is sleep need (which can change dependent upon other measured parameters, or upon stored data, or can be set manually).

The device 1, being a highly portable unit, may easily be taken periodically, typically fortnightly to an expert sleep analyst for further interrogation and more detailed advice. A download or transmission facility is provided within the device to enable the data to be extracted for this interrogation process.

The sleep expert will provide a more detailed analysis of the user's sleep patterns. For example, in order to provide a measure of sleep quality, as described above, a parameter “sleep quality index” (SQI) may be provided. The algorithm for this parameter SQI, is based upon many of the parameters which are easily monitored, or derived by the invention. The algorithm is

$$SQI = C + {\sum }_{i = 1}^{n} {C}_{i} {P}_{i}$$

This algorithm uses twelve parameters and their associated constants (i.e. n=12). The parameters are: Time in Bed*, Sleep Efficiency, Mean Wake Bout Time, Sleep End Time, Sleep Latency, Mean Activity Score, Actual Sleep Time, Sleep Bouts, Mean Score Inactive*, Actual Sleep (%), Wake Bouts, and Wake Movement RMS.

Corresponding constants may be defined by the values as follows:

| | |
|---|---|
| Constant (C) | 52.42 |
| Time in bed (C₁)* | −1.887 |
| Sleep end time (C₂)* | 0.572 |
| Actual sleep time (C₃) | 2.084 |
| Actual sleep (%) (C₄) | −0.3536 |
| Sleep efficiency (C₅) | 0.1408 |
| Sleep latency (C₆) | −0.018 |
| Sleep bouts (C₇) | 0.188 |
| Wake bouts (C₈) | −0.2469 |
| Mean wake bout time (C₉) | −1.2126 |
| Mean activity score (C₁₀)* | −0.226 |
| Mean score inactive (C₁₁)* | −0.0112 |
| Wake movement RMS (C₁₂) | 0.001238 |

Alternatively, this algorithm may be customised to represent an individual user, to achieve results of greater accuracy.

Solution of this algorithm can be intensive in terms of processing requirements. Where the processing capacity is not extensive, as in the analysing means 17 of the device 1, a simpler formulation may be implemented. In this case, a value for quality of sleep is estimated by the user and this value is modified, based on four of the monitored/derived parameters (those marked ) above. This basic interpretation of sleep quality gives a lower predictive accuracy, nevertheless it provides a useful gauge, on a day to day basis, for the user of the device.

Alternatively, the quality of the sleep may be represented by a parameter which may be biased by user supplied estimates. The parameters used in the biasing process may be time in bed, sleep end time, mean activity score and/or mean score inactive.

The invention is described for use in monitoring sleep patterns, however, it may also be of use in monitoring alternative body activities. For example monitoring daily activity levels to indicate whether the user is achieving sufficient activity in a fitness regime, while on a diet, during recuperation or, when bed rest is necessary, the level of activity of a patient determines whether bed sores will be prevented. A further example may be to study the activity of children who suffer attention deficit syndrome. There are many other scenarios where the standard equipment could be used to monitor the activity of people or even pets.

If further sensors were introduced such as a heart rate sensor the device could be used to monitor the heart rate either during sleep, to determine the different phases of sleep or during sports activities to monitor the heart rate without the need for any cumbersome chest band. The device could also be used to determine how stressed somebody was and potentially warn of impending heart problems.

In an alternative embodiment of the invention, there is a method for monitoring other body activities. Again, the invention comprises a method having the steps of receiving actimetry data from a sensor measuring body activity, analysing the data to provide advisory information, and determining parameters based upon the actimetry data. The parameters are combined to generate the advisory information, including an indicium of the quality of the activity according to the following relationship:

$$AQI = C + {\sum }_{i = 1}^{n} {C}_{i} {P}_{i}$$

wherein AQI is the activity quality index and is an integer greater than 1, Cᵢ is a set of constants, and Pᵢ is a set of parameters. The parameters include at least one parameter selected from the group consisting of activity duration, activity efficiency, activity latency, activity bouts, the number of activity interruptions, and the mean activity score. C is again a constant. Thus, the body activity being monitored may be a sleep activity and/or a waking activity.

In a further example, the additional analysing means may or may not be provided. In this case, the receiving device (or docking station) may be configured to perform the analysis procedure, while the level of analysis would necessarily be less comprehensive than the analysis means, due to the reduced processing capacity, the unit still provides a useful function to the user. The docking station may be provided with a display and controls to enable the user to select and display information from the analyses performed by the unit. A recess may be provided to locate the sensor device in the correct relative position to assist in downloading the information stored within it.

In use, the sensor device will be initialized in the docking station prior to use (i.e., before bed time). When the user wishes to retire, the sensor device will be removed from the docking station and placed on the wrist. The docking station comprises its own internal clocking mechanism and hence removing the sensor device from the recess on the docking station will automatically provide “bed time” information. Similarly, “wake time” will automatically be recorded when the sensor device is replaced into the recess of the docking station the following morning. This facility reduces the need for the user to keep a paper “sleep diary” and consequently makes the system easier to use.

Alternatively, the monitoring device may not comprise a storage facility. In this case, the data acquired by the actimetry sensor would be transmitted directly to the docking station for storage in real time.

Provision of a more sophisticated docking station may eliminate the need for a user to have a computer available to perform the analyses. However, the level of analysis achieved, as described above, may be less comprehensive. The docking station may be a highly portable unit that may easily be taken periodically, typically fortnightly, to an expert sleep analyst for further interrogation and more detailed advice. A download facility is provided within the docking station to further assist in this interrogation process.

Introduction of a global positioning system in combination with the actimetry sensor would allow the device to be used to track the whereabouts and activity of children, old people (particularly Alzheimer's patients) or perhaps criminals on probation. If the actimetry sensor were used in combination with a clock, the device could be used to help controlling jet lag by recommending the best sleeping habits to cope with a particular difference in time zone.

While particular embodiments of the present invention have been illustrated and described, it would be obvious to those skilled in the art that various other changes and modifications can be made without departing from the spirit and scope of the invention. Any of the aspects of the invention of the present invention found to offer advantages over the state of the art may be used separately or in any suitable combination to achieve some or all of the benefits of the invention disclosed herein.

What is claimed is:

1. A device for monitoring body activity, the device comprising:
an actimetry sensor for measuring body activity;
storage means for receiving data from the actimetry sensor;
means for analysing the data to provide advisory information using a summation of a plurality of parameters, said parameters relating to body activity information; and
means for displaying the advisory information.

2. The device of claim 1, wherein the actimetry sensor comprises an accelerometer.

3. The device of claim 2, wherein the advisory information provides an indication of, the quality of sleep of a user.

4. The device of claim 3, further comprising storage means to store data from the actimetry sensor.

5. The device of claim 4 further comprising means to store temporal information.

6. The device of claim 5, wherein the means for analysing the stored data analyses said body activity information and said temporal information.

7. The device of claim 1 further comprising a transmitter for transmitting data from the monitoring device to an external receiver.

8. The device of claim 7, wherein said transmitter transmits said data to a computer readable storage medium.

9. The device of claim 3 capable of monitoring at least one sleep phase selected from the group consisting of REM, slow light sleep, slow deep sleep, and paradoxical sleep.

10. The device of claim 2, further comprising at least one additional sensor for measuring at least one of body pulse rate and/or blood pressure.

11. The device of claim 10, further comprising an alarm, said alarm providing an indication of a said body activity falling above or below a threshold level.

12. The device of claim 10, further comprising a sensor for detecting data relating to the environment in which said device is placed.

13. A method for monitoring body activity, said method comprising the steps of:
providing an actimetry sensor for measuring a body activity;
juxtaposing said actimetry sensor with a body, whereby said actimetry sensor can measure said body activity;
performing an activity;
receiving data from said actimetry sensor relating to said activity, said data comprising a plurality of parameters; and
analysing said data to provide advisory information for a user, wherein said analysis includes the steps of summing a plurality of said parameters relating to said activity.

14. The method according to claim 13, wherein at least one of said parameters is selected from the group consisting of time in bed, sleep end time, actual sleep time, actual sleep (%), sleep efficiency, sleep latency, sleep bouts, wake bouts, mean activity score, mean score inactive, mean wake bout time, and wake movement RMS, whereby said analysis provides an indication of the quality of sleep of a user.

15. The method according to claim 13, wherein said step of summing said parameters includes summing parameters indicating time in bed, sleep end time, mean activity score, and mean score inactive.

16. A method according to claim 15, wherein twelve parameters are used in said analysis, according to the equation
$$SQI = C + {\sum }_{i = 1}^{12} {C}_{i} {P}_{i}$$
wherein SQI represents a sleep quality index, wherein C represents a constant, Cᵢ represents a constant associated with each said parameter, and Pᵢ represents a parameter relating to sleep.

17. The method according to claim 16, wherein said analysis utilizes the constants of C equals 52.42, C₁ equals −1.887, C₂ equals 0.572, C₃ equals 2.084, C₄ equals −0.3536, C₅ equals 0.1408, C₆ equals −0.018, C₇ equals 0.188, C₈ equals −0.2469, C₉ equals −1.2126, C₁₀ equals −0.226, C₁₁ equals −0.0112, and C₁₂ equals 0.001238.

18. The method according to claim 13, wherein the quality of the activity is analysed according to a user supplied estimate of the parameters.

19. A method for monitoring body activity, the method comprising the steps of:
receiving actimetry data from a sensor measuring body activity;
analysing the actimetry data to provide advisory information, wherein the analysing step comprises the steps of:
determining parameters based on the actimetry data; and
combining these parameters to generate advisory information including a measure of the quality of the activity according to the following relationship:
$$AQI = C + {\sum }_{i = 1}^{n} {C}_{i} {P}_{i}$$
where AQI is the “activity quality index”, n is an integer greater than 1, C₁ is a set of constants and Pᵢ is a set of parameters, at least one of said parameters being selected from the group consisting of activity duration, activity efficiency, activity latency, activity bouts, number of activity interruptions and mean activity score; and
outputting said advisory information.

20. A method according to claim 19, wherein said advisory information further includes at least one piece of information selected from the group consisting of comparative data to peer group data, statistical analysis, history data and solution/product choice.
